(19) United States
(12) Patent Application Publication
(10) Pub. No.: US 2010/0185267 A1
(43) Pub. Date: Jul. 22, 2010

(54) METHOD AND APPARATUS FOR CONTROLLING MENOPAUSAL HOT FLASHES

(76) Inventor: Robert G. Dickie, King City (CA)

Correspondence Address: SAND & SEBOLT, AEGIS TOWER, SUITE 1100, 4940 MUNSON STREET, NW, CANTON, OH, 44718-3615, US

(21) Appl. No.: 12/357,431

(22) Filed: Jan. 22, 2009

(51) Int. Cl. A61F 7/02

(52) U.S. Cl. 607/109

(57) ABSTRACT

A device and method for non-invasive treatment of menopausal hot flashes includes a housing with an anatomically configured bottom wall. A Peltier effect device and heat sink are in thermal contact with a cold plate that extends from the bottom wall. As the cold plate is moved back and forth across the skin it stimulates cold thermoreceptors which signal the hypothalamus and counteract the false trigger which caused the hot flash. The cold plate, which is cooled to a temperature of around 10 degrees Celsius, is left in contact with a region of skin for no more than 10-15 seconds so that the thermoreceptors do not become saturated. The device shuts off after 1 minute.

### BACKGROUND OF THE INVENTION

1. Technical Field

This invention generally relates to therapeutic devices. More particularly, the invention relates to a device that is useful for controlling or stopping a menopausal hot flash. Specifically, the device includes at least one cold plate that is cooled using a Peltier effect device and is moved back and forth across the back of the neck to stimulate cold thermoreceptors in the skin and to thereby counteract false triggering of the hypothalamus that causes a hot flash.

2. Background Information

Mammals are warm blooded creatures that are able to thrive in a wide range of environments for a number of reasons. One of these reasons is that that their bodies are provided with complex thermoregulatory systems that enable them to monitor the ambient temperature, i.e., the temperature of their surroundings, and to adjust various bodily functions to keep their internal core temperature substantially constant. The core temperature is vital to the survival of the mammal in that if it becomes too high they will die and if it drops too low they will die. If the ambient temperature increases, the mammal's body will tend to absorb some of this surrounding heat and their core temperature will tend to rise in response. Similarly, if the ambient temperature drops, the body will naturally radiate heat into the environment thereby causing a drop in the body's core temperature. The thermoregulatory system will adjust body functions so as to lose heat, prevent heat loss or even generate heat in order to keep the core temperature more or less constant.

In humans, who are mammals, one of the most important parts of this thermal monitoring and regulation system is the skin. Skin includes both hot and cold temperature receptors that detect the ambient temperature and then fire a signal to the hypothalamus. The hypothalamus takes action in response to this data and changes various bodily functions to regulate the core temperature. These bodily functions controlled by the hypothalamus include, but are not limited to, the body's heart rate, the rate of breathing, the extent of blood flow to the extremities, and rates of sweating and shivering If, for example, the thermoreceptors in the skin indicate that the environment is too hot and that the core temperature is therefore likely to rise, the heart rate will be increased and blood flow to the capillaries that are just beneath the skin's surface will be increased. Both of these actions bring warmed blood closer to the skin very rapidly so that heat can radiate from the blood to the environment. Additionally, the sweat glands are stimulated and liquid is released onto the skin's surface. Evaporation of this liquid utilizing heat from the blood close to the skin's surface causes the temperature of the skin, the blood and therefore the body's core to be lowered. If, on the other hand, the receptors detect that the environment is too cold and that the core temperature is therefore in jeopardy of falling too low, the thermoregulatory system will slow the heart rate and will decrease blood flow to the body's extremities which causes capillaries close to the skin's surface to close. This slows down the rate at which heat is radiated into the environment from the body. Furthermore, hair follicles on the skin's surface are stimulated to vibrate, a condition typically referred to as shivering. This generates heat which flows into the blood. These actions attempt to keep the body's core temperature in a safe range.

As any menopausal woman knows, one of the most unpleasant symptoms of this time of life is the disruption in the body's complex thermoregulatory system. There is periodically a false triggering of the thermoregulatory system which affects or is internal to the hypothalamus. This false trigger causes the hypothalamus to determine that the body's core temperature is rising rapidly and is getting dangerously high. The hypothalamus responds by taking the action that is typically required to lower the core temperature. This response includes vascular dilation and an increase in heart rate to get blood more rapidly to the skin for cooling. These actions cause the woman to feel flushed, out of breath and extremely hot as the blood is rushed to the skin. In other words, the woman has a hot flash. The result of this hypothalamic action is that the body's core temperature drops by as much as two to three degrees Celsius. Once the hot flash is over, the woman may experience chills for an hour or more as her body attempts to build back up to a safer core temperature range While hot flashes cause the body temperature to rise rapidly for one or two minutes and are then gone, these extreme temperature swings can leave the woman drained and emotionally distraught. While this condition is hormonally based and is therefore extremely difficult to avoid or correct, it is desirable that at least the symptoms thereof be addressed in some manner that will bring the menopausal woman relief and leave her with a greater sense of being in control of her own body.

There is therefore need in the art for method of controlling the menopausal symptoms commonly known as hot flashes and for a device that is used for this purpose. The present inventor has recognized that the body's own thermoregulatory system may be utilized to aid in reducing the menopausal symptoms known as hot flashes. Furthermore, the inventor has recognized that the skin plays a large role in assisting to keep the core temperature of the body more or less constant. Even further, the inventor has recognized that the skin at the back of the neck has a very high density of thermoreceptors and that it is a key area of the body that the hypothalamus monitors in determining ambient temperature. While the present inventor is unaware of any devices or modalities that are used to control menopausal symptoms using the body's thermoregulatory system in the manner proposed in this specification as follows, the prior art has disclosed various devices and modalities for the treatment of other ailments that utilize neural pathways. Once such device and modality is disclosed in U.S. Pat. No. 5,628,769 to Saringer. Saringer's device is used in the treatment of intense localized pain and includes a mechanism for creating a spatial temperature differential in one of the device's surfaces. The temperature differential is set up in a surface area that is around 1 square centimeter in size, and the mechanism generates an intensely high temperature in a first region of this surface and an intensely low temperature in a second region of this surface. The high temperature is maintained at around 45 degrees Centigrade and the low temperature is maintained at around 0 degrees Centigrade. The temperature differential is therefore around 45 degrees Centigrade across this 1 square centimeter. This small surface area is then placed in contact with the patient's skin in the immediate area experiencing pain and is kept in place for a period of 15 to 20 minutes. The sensation generated by the device is felt by the patient as intense heat. The temperature differential generates a large neural signal that travels along much the same neural pathways as the pain signals would travel, and the pain signals are effectively blocked by this device.

### SUMMARY OF THE INVENTION

The device of the present invention comprises a hand-held unit that includes a housing with a solid state cooling system disposed therein. A cold plate is mounted on the housing and a least a portion thereof extends outwardly away therefrom. The cold plate is operationally connected to the cooling system and the entire cold plate is cooled by the cooling system. The cold plate is brought into abutting contact with the skin at the back of a woman's neck and is moved back and forth thereacross. The cold plate is not kept in any one position for a period of longer than 10 to 15 seconds. After 1 minute the device automatically shuts off.

Inasmuch as during a hot flash the hypothalamus has been falsely triggered into determining that the body's core temperature is rising, the device of the present invention is designed to specifically counteract that false trigger. This is accomplished by the device being used to cool and thereby stimulate the cold thermoreceptors in the skin at the back of the neck. In response to being cooled, the thermoreceptors fire and emit a signal to the hypothalamus indicating that the ambient temperature is extremely cold. In response to this signal, the hypothalamus stops trying to decrease the core temperature of the body. Consequently, the woman's heart rate slows and blood is no longer rushed to the surface of the skin for cooling. Thus, the false trigger is counteracted and the hot flash effectively ceases. The cold plate is kept in any one position for no longer than 10 to 15 seconds as this is the time it typically takes for the thermoreceptors to become saturated and stop emitting a signal to the brain. Since a hot flash typically lasts only for two to three minutes, the cold thermoreceptors need only be stimulated for a very short time in order to cause the hot flash to cease. Furthermore, since the hot flash cycle is interrupted, the body's core temperature does not drop and there is therefore no need for the body to try and raise the core temperature once again. Consequently, the subsequent adverse effects of the hot flash are also averted by using the device of the present invention.

### BRIEF DESCRIPTION OF THE DRAWINGS

The preferred embodiments of the invention, illustrative of the best mode in which applicant has contemplated applying the principles, are set forth in the following description and are shown in the drawings and are particularly and distinctly pointed out and set forth in the appended claims.

FIG. 1 is a top perspective view of the device of the present invention showing the upper and lower sections of the housing;

FIG. 2 is a bottom perspective view of the device in accordance with the present invention and showing the upper and lower sections of the housing;

FIG. 3 is an exploded perspective view of the device of FIGS. 1 and 2;

FIG. 4 is a top view of the upper section of the device;

FIG. 5 is a cross-sectional side view of the device taken through line 5-5 of FIG. 4;

FIG. 6 is a side view of the device showing the recharging connector on the upper section of the housing;

FIG. 7 is a bottom view of the device showing the anatomically shaped bottom wall and the cold plates mounted therein;

FIG. 8 is a rear view of a woman applying the device to the back of her neck;

FIG. 9 is an enlarged rear view of the device on the woman's neck with the woman's hands removed for clarity and showing the cold plates in phantom;

FIG. 10 is a rear view of the woman's neck showing the device being moved in a first direction across the neck from a first position to a second position; and

FIG. 11 is a rear view of the woman's neck showing the device being moved in a second direction across the neck and from the second position to a third position.

### DETAILED DESCRIPTION OF THE INVENTION

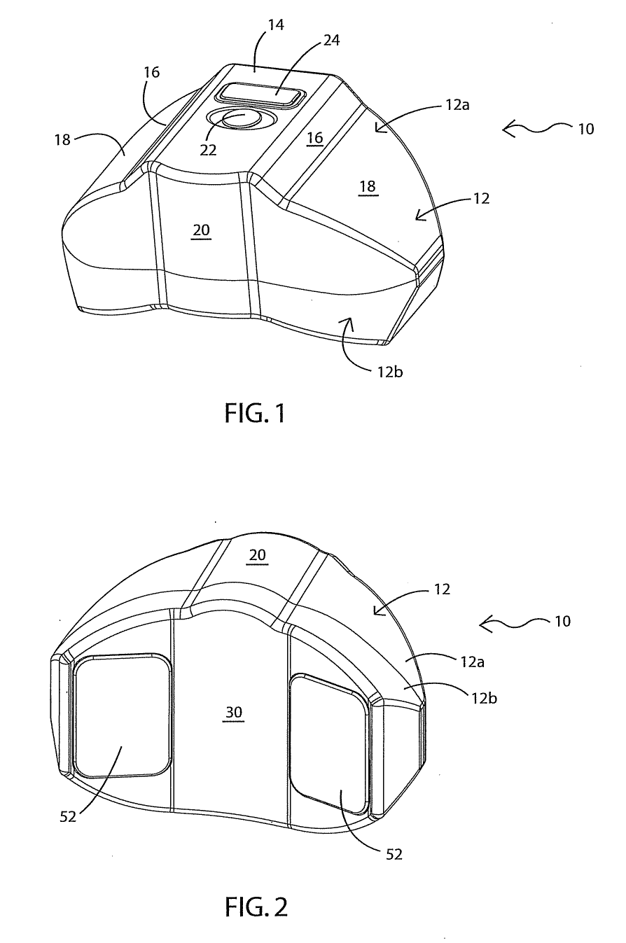

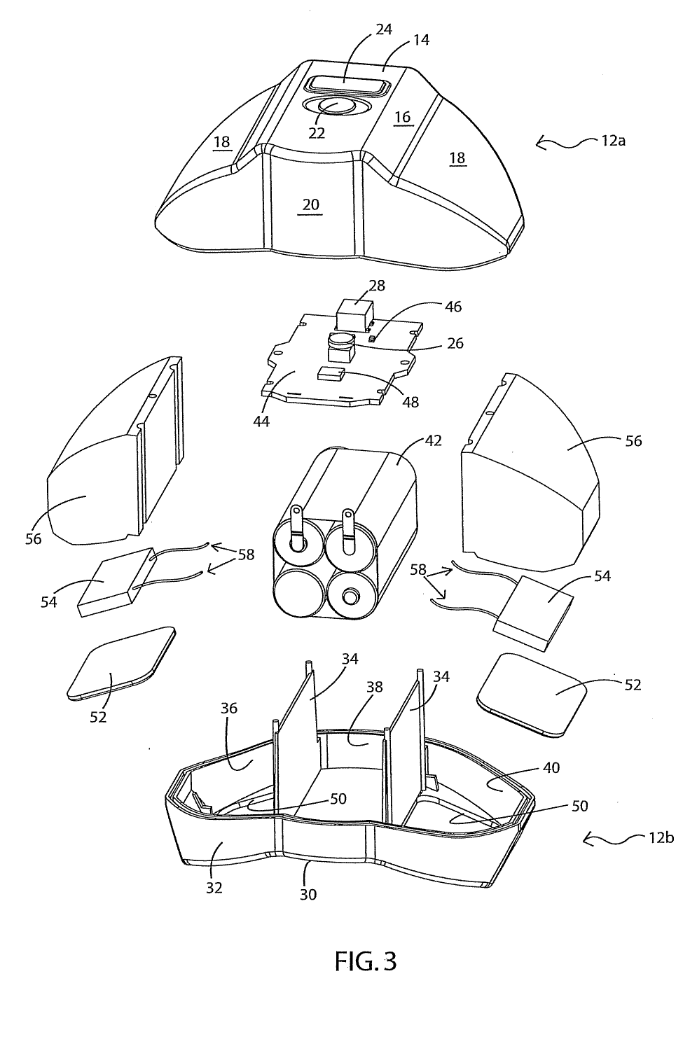

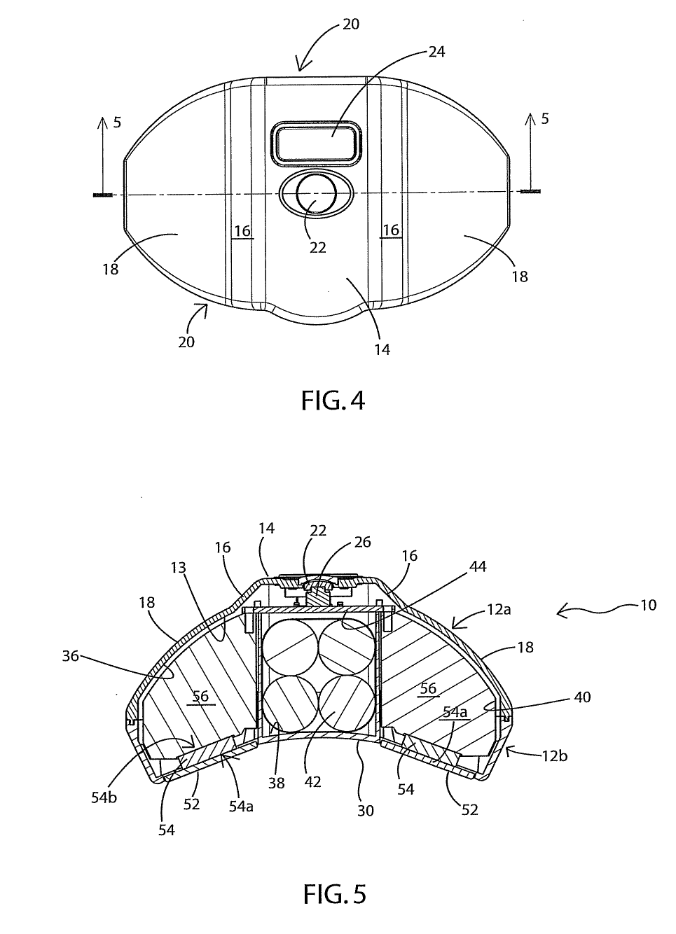

Referring to FIGS. 1-7 there is shown a medical device in accordance with the present invention and being generally indicated at 10. Device 10 comprises a housing 12 made up from an upper section 12a and a lower section 12b that are interlockingly engaged with each other. Each of the upper and lower sections 12a, 12b preferably is molded from any suitable material such as Acrylonitrile Butadiene Styrene plastic (i.e., ABS plastic) and the sections are configured to snap fit together. Upper and lower sections 12a, 12b are configured to define an interior chamber 13 (FIG. 4) which houses a plurality of components as will be hereinafter described. When device 10 is assembled, lower section 12b is configured to be brought into abutting contact the back of a patient's neck and the upper section is configured to be easily gripped in the palm of one hand.

Upper section 12a includes an exterior wall comprising a raised central portion 14, two transition zones 16 sloping sharply downwardly from central portion 14, and two exterior surfaces 18 that slope downwardly away from the transition zones 16 and in opposite directions from each other. The contoured shape of upper section 12a helps the device 10 to be easily seated within the palm of one hand. Transition zones 16 provide suitable areas on device 10 for a person to place their thumb and fingers should they wish to grip the device in that manner. Additionally, the side walls 20 of upper section 12a are configured to enable the user to easily grip device 10 between their thumb and fingers if they wish to grip the device in that manner. Upper section 12a is provided with buttons 22, 24 thereon for operating an on/off switch 26 and a recharging connector 28 that are disposed within the interior chamber 13 of housing 12.

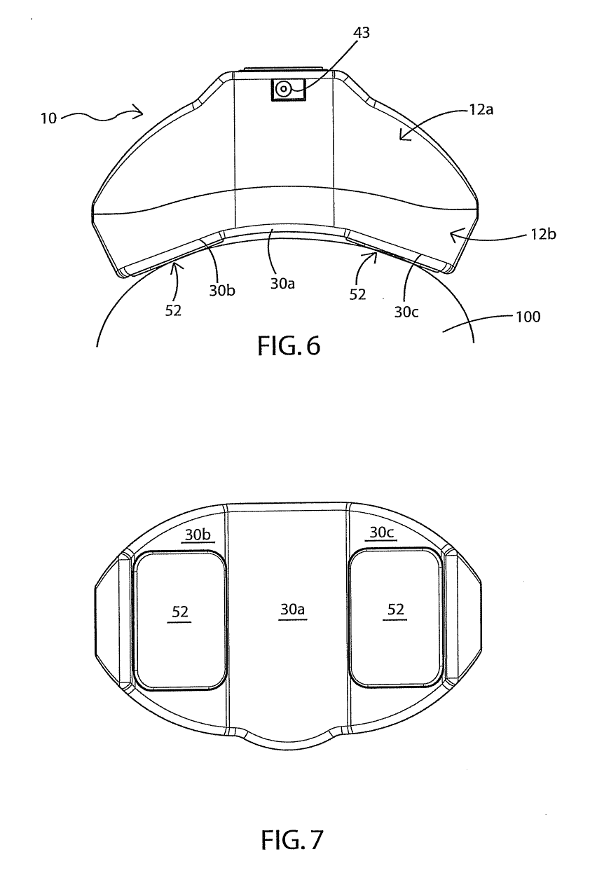

Lower section 12b of housing 12 includes a bottom wall 30 and a perimeter wall 32 that extends upwardly away from bottom wall 30. Perimeter wall 32 is shaped to interlockingly engage portions of side walls 20 and exterior surfaces 18. Bottom wall 30 is configured to be anatomically shaped to fit the rear curvature of a person's neck. FIGS. 6 & 7 show that bottom wall 30 has a central region 30a flanked by two side regions 30b, 30c that angle downwardly away from central region 30a. This shape permits bottom wall 30 to remain in abutting contact with the back of the neck as device 10 is moved thereacross, as will be hereinafter described.

The interior surface of bottom wall 30 preferably includes one or more dividers 34 that extend upwardly away therefrom. These dividers 34 separate lower section 12b into compartments 36, 38, 40 which make it easier to house the operating components of device 10. These components include a power source 42 that is retained within compartment 38. In the preferred embodiment of the invention, the power source comprises one or more rechargeable NiCad batteries. Preferably, power source 42 comprises four rechargeable NiCad batteries. FIG. 6 shows a rear view of device 10 and shows the connector 28 into which one of a remote recharging unit (not shown) or AC supply may be operationally connected in order to recharge the batteries. It will be understood that any other suitable power source may, alternatively, be utilized in device 10 other than the rechargeable batteries.

Device 10 is also provided with a circuit board 44 in chamber 13. Circuit board 44 includes the on/off switch 26, the recharging connector 28, a LED light 46 to indicate whether the device is on or off, and a microprocessor 48. Microprocessor 48 controls device 10 and includes various thermal protection override features and safety features. Microprocessor 48 monitors the battery charge level and automatically shuts off device 10 if any operational issues arise. The electrical circuitry that connects circuit board 44 to the other components within housing 12 is not shown for the sake of clarity.

In accordance with a specific feature of the present invention, lower section 12b defines at least one and preferably two apertures 50 defined in bottom wall 30. Preferably each aperture 50 is defined in one of the regions 30b, 30c of bottom wall 30. Apertures 50 open into the interior chamber 13 and specifically into compartments 36 and 40, respectively. Device 10 further includes at least one, and preferably two cold plates 52. Each cold plate 52 is fixedly mounted within one of apertures 50 in such a manner that the cold plates 52 extend slightly outwardly beyond the regions 30b, 30c of bottom wall 30 as shown in FIG. 5. Each cold plate 52 preferably is substantially rectangular in shape and is around 1″×1.5″ in size. Central region 30a is of a width that is greater than the width of each plate 52. Thus, central region 30a is of a width that is greater than one of 1″ and 1.5″, depending on the orientation of plates 52. The entire surface area of cold plates 52 that extends outwardly from beyond bottom wall 30 is designed to contact the patient's skin. In the preferred embodiment of the invention, cold plates 52 are manufactured from aluminum, but it will be understood that plates 52 may be made from any thermally conductive material.

In accordance with yet another specific feature of the present invention, device 10 is provided with a solid state cooling system which includes one, but preferably two, Peltier effect elements 54 and one, but preferably two, heat sinks 56. In the preferred embodiment, heat sinks 56 are manufactured from aluminum but they may alternatively be manufactured from copper or any other suitable material known in the art. A first Peltier element 54 and heat sink 56 is disposed in compartment 36 and a second Peltier element 54 and heat sink 56 is disposed in compartment 40. Each Peltier element 54 is disposed between one of the cold plates 52 and one of the heat sinks 56. The Peltier elements 54 are in thermal contact with the associated cold plate 52 and heat sink 56. If elements 54 are in direct abutting contact with cold plates 52 and heat sinks 56, a suitable thermal grease may be applied therebetween as is known in the art. Each Peltier element 54 is electrically connected to power source 42 by a pair of wires 58. The Peltier elements 54 are provided as solid state heat pumps that are used to lower the temperature of the entire cold plates 52 or at least the entire skin-contacting surface of the cold plates 52. In the preferred embodiment, Peltier elements 54 lower the temperature of cold plates 52 to a temperature of around 10 degrees Celsius. This is about ten degrees lower than a normal ambient temperature of 20 degrees Celsius. It will be understood that the microprocessor could be programmed to cause the Peltier effect devices 54 to cool cold plates 52 to temperatures lower or higher than 10 degrees Celsius if this was desirable or necessary for the device to function in the manner contemplated. The temperature of cold plates 52 needs to be sufficiently low enough to stimulate cold thermoreceptors in the skin at the back of the neck to send a signal to the hypothalamus.

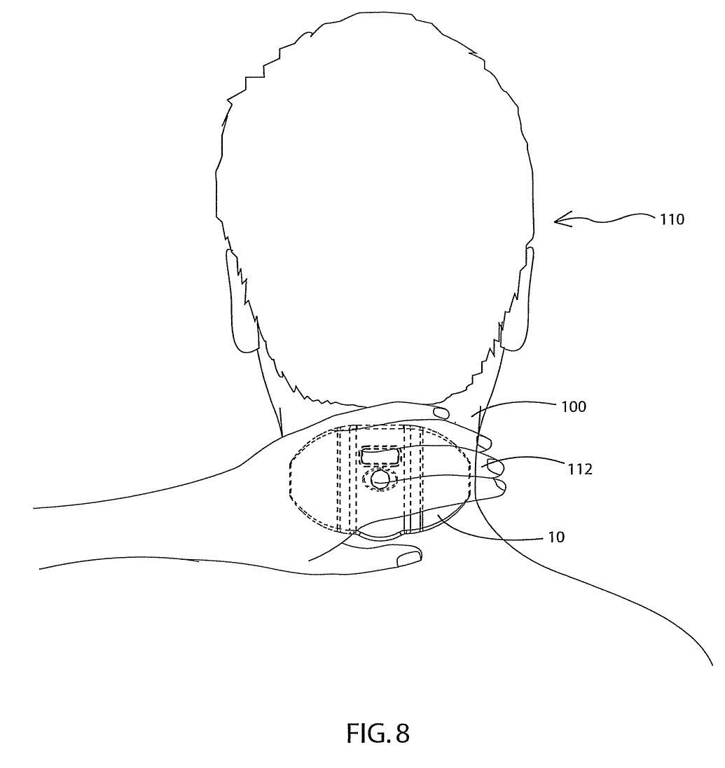

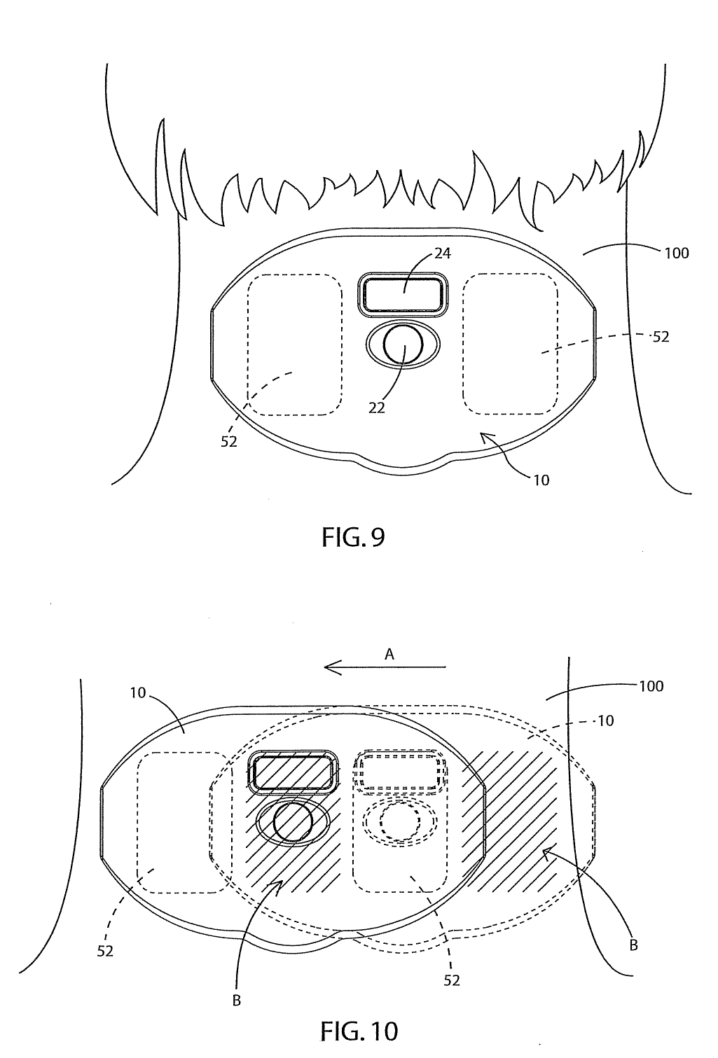

Referring to FIGS. 8-11, the device 10 is used in the following manner on the skin on the back of the neck 100 of a woman 110. When the woman 110 feels the onset of a hot flash, she depresses the button 22 on upper section 12a thereby activating the on/off switch 26 for device 10. This activation applies a voltage across the Peltier effect devices 54 causing a rapid drop in temperature of the surfaces 54a (FIG. 5) thereof that are in contact with cold plates 52. Surfaces 54a, in turn, cool cold plates 52 in a time period of between three and five seconds. The woman 110 then holds device 10 in her hand 112 and places bottom wall 30 of device 10 in contact with the back of her neck 100 as is illustrated in FIG. 8. FIG. 9 shows the top of device 10 with cold plates 52 shown in phantom and with the woman's hand removed therefrom for the sake of clarity. It will be understood that the skin 100 situated in the region of the neck that is in abutting contact with cold plates 52 is cooled by contact with cold plates 52. This causes the thermoreceptors in the skin to be stimulated to a degree sufficient to emit a signal to the nervous system. Heat from the skin 100 is conducted into cold plates 52 and via Peltier elements 54 to heat sinks 56. Device 10 is held in this first position shown in FIG. 9 until the cold sensation on the neck begins to subside. This subsidence of the sensation occurs as the thermoreceptors are stimulated to a maximum degree, a condition known as saturation. Once saturation is reached, the thermoreceptors will no longer emit a signal to the brain. In order to prevent the cessation of a signal to the brain, the woman 110 must move device 10 on her skin 100 from the first position to a second position. This is illustrated in FIG. 10 where device 10 is moved in a first direction A across skin 100 to a second position. In this second position, cold plates 52 are disposed in contact with a new region of skin in which the thermoreceptors have not yet been stimulated. The previously cooled regions of skin that were cooled when the device was in the first position are illustrated in FIG. 10 as areas B. The thermoreceptors in areas B will reset themselves after a time period of about five to ten seconds. This means that, after a five to ten second period, the thermoreceptors are once again in a condition where they may be stimulated and will once again emit a signal to the brain.

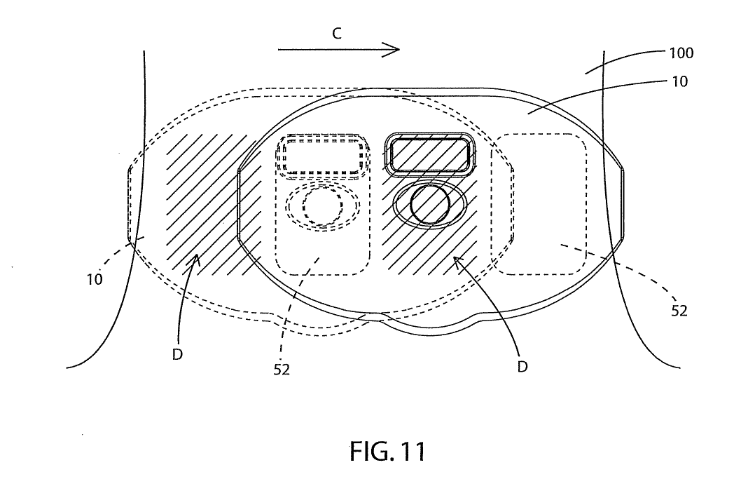

The temperature of cold plates 52 on device 10 remains substantially constant because of the thermal transfer of heat from plates 52 through the Peltier effect device 54 to heat sinks 56. Consequently, when device 10 has been moved in direction A, the new regions of skin 100 that are disposed beneath the cold plates 52 when device 10 is in this second position are cooled by contact with plates 52. When the cold sensation on the neck begins to subside once again because the thermoreceptors in this second region have become saturated, the woman 110 must move device 10 to a third position in a different location. Accordingly, as is shown in FIG. 11, device 10 may be moved in a second direction C so that cold plates 52 are disposed in contact with a new region of skin 100. This new region of skin may be region B, where the thermoreceptors have been automatically reset by the body, or may be a totally different region of skin. The regions of skin that were cooled when device 10 was in the second position shown in FIG. 10 are indicated in FIG. 11 as regions D. Once cold plates 52 have been removed from regions D, the thermoreceptors in the skin in those regions are reset by the body after five to ten seconds. The cold plates 52 are spaced a distance away from each other on bottom wall 30. This balances the desirability of providing the greatest possible area of contact between the cold plates 52 and the skin while still providing sufficient areas of the skin that are not contacted by the plates 52 so that the thermoreceptors in the previously cooled areas have sufficient time to reset without a substantial drop in the neural signal strength to the brain.

The woman 110 keeps “shuttling” the device 10 back and forth in this manner, holding the device in each new position until the cold sensation of the plates on the skin begins to subside before she changes the device's position on the back of the neck. The woman 110 will typically have to relocate the device 10 every ten to fifteen seconds in order to have device 10 contact “new” skin where the skin's thermoreceptors have not been stimulated or have already been reset. This shuttling motion helps to keep the thermoreceptors in the skin firing signals to the hypothalamus at a maximum rate. After a time period of around sixty seconds, device 10 automatically shuts off. This time period of activation has been found to be sufficient to stop the symptoms of a hot flash before they are fully expressed. If the hot flash ceases before the automatic shutoff of the device 10, the woman 110 may simply depress button 22, thereby depressing the on/off switch 26 and deactivating device 10.

While the preferred embodiment of the present invention has been described as having two cold plates that are each in abutting contact with a separate Peltier effect device, which in turn are in operational contact with a separate heat sink, it will be understood that the device may include one or more cold plates that are in contact with a single Peltier effect device that is in turn in operational connection with a single heat sink. Furthermore, the device may include more than one cold plate, each of which is in contact with its own Peltier effect device, and that the more than one Peltier effect devices may be in operational contact with a single heat sink. The device may further include other components that aid in dissipating heat from the heat sink such as openings in the housing, fans etc.

Microprocessor 48 monitors the temperature of various components within device 10, and more specifically the temperature of heat sinks 56. If the monitored temperatures are above a preset, pre-determined level, then microprocessor 48 will prevent activation of device 10.

It will be understood that while the device of the present invention has been described specifically for reducing the effects of menopausal hot flashes, the device could be used as a heated massage device for the neck. Peltier effect devices are constructed in such a manner that one surface, such as surface 54a (FIG. 5), becomes cooled and the opposite surface 54b becomes heated. The determination as to which of the two surfaces 54a, 54b is heated or cooled results from the polarity across the device 54. So, for example, if one wished to heat plate 52 instead of cooling the same, the DC polarity across the Peltier effect device 54 could be reversed. Plate 54b would then become cooled and plate 54a would become heated, thus heating plate 52 and thereby the skin within which that plate is in contact.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A device for the non-invasive treatment of menopausal hot flashes, said device comprising:
a housing;
a cooling system disposed within the housing;
a first thermally conductive plate extending at least partially outwardly from the housing; said first plate being thermally connected to the cooling system so as to be entirely cooled thereby; and wherein said first plate includes a surface that is adapted to be brought into abutting contact with the skin at the back of a woman's neck.

2. The device as defined in claim 1, wherein the cooling system is a solid state cooling system that includes:
a power source; and
at least one Peltier effect device electrically connected to said power source.

3. The device as defined in claim 2, wherein the cooling system further includes:
at least one heat sink thermally connected to said at least one Peltier effect device.

4. The device as defined in claim 1, wherein said housing comprises an upper portion and a lower portion, and wherein said lower portion has a bottom wall that is configured to be anatomically shaped to abut the back of a person's neck.

5. The device as defined in claim 4, wherein the bottom wall includes a central region with two side regions extending outwardly away from the central region; and wherein the at least one cold plate is mounted on one of said side regions.

6. The device as defined in claim 1, wherein said housing comprises an upper portion and a lower portion, and said lower portion has a bottom wall that is configured to be anatomically shaped to abut the back of a person's neck; and wherein the first plate is mounted in the bottom wall; and the device further comprises a second thermal plate mounted in the bottom wall a spaced distance from the first cold plate.

7. The device as defined in claim 6, wherein each of the first and second cold plates is substantially rectangular in shape and is around 1″×1.5″ in size.

8. The device as defined in claim 7, wherein the first and second plates are disposed a distance away from each other and that distance is greater than the width of either of the first and second plates.

9. The device as defined in claim 6, wherein the cooling system is a solid state cooling system that includes:
a power source; and
a first Peltier effect device electrically connected to said power source.

10. The device as defined in claim 9, wherein the first Peltier effect device is thermally connected to the first plate; and the cooling system further includes a second Peltier effect device that is thermally connected to the second plate; and a first heat sink that is thermally connected to one or both of the first and second Peltier effect devices.

11. The device as defined in claim 10, wherein the cooling system further includes a second heat sink, and the first heat sink is thermally connected to the first Peltier effect device and the second heat sink is thermally connected to the second Peltier effect device.

12. The device as defined in claim 11, wherein the first and second heat sinks are manufactured from aluminum.

13. The device as defined in claim 6, wherein each of the first and second plates is manufactured from aluminum.

14. The device as defined in claim 2, further including a microprocessor that is operationally connected to the cooling system.

15. A method of controlling menopausal hot flashes in a woman's body comprising the steps of:
a) activating a therapeutic device to cool a thermally conductive cold plate in the device at the first sign of a hot flash;
b) bringing the cold plate into abutting contact with a first region of skin at the back of the woman's neck;
c) holding the cold plate in contact with the first region of skin for a first period of time;
d) moving the device along the skin until the cold plate contacts a second region of skin at the back of the woman's neck remote from the first region of skin; and
e) holding the cold plate in contact with the second region of skin for a second period of time.

16. The method as defined in claim 15, further comprising the steps of:
f) moving the device along the skin until the cold plate contacts one of a third region of skin at the back of the neck and the first region of skin; and
g) holding the cold plate in contact with the one of the first and third regions of skin for a third period of time.

17. The method as defined in claim 16, further comprising the steps of repeating steps d) through g) until one of the device automatically shuts off and the hot flash ceases.

18. The method as defined in claim 15 wherein each of the first and second periods of time are between ten and fifteen seconds long.

19. The method as defined in claim 16, wherein the third period of time is between ten and fifteen seconds long.

20. The method as defined in claim 17, wherein the device automatically shuts off after 1 minute.

21. The method as defined in claim 15 further comprising the step of waiting for the cold plate to cool to a temperature of about 10 degrees Celsius prior to bringing the cold plate into contact with the skin.

22. The method as defined in claim 21, wherein the step of waiting for the cold plate to cool takes between three and five seconds.

23. A therapeutic device comprising:
a housing;
a heating and cooling system disposed within the housing;
a first thermally conductive plate extending at least partially outwardly from the housing; said first plate being thermally connected to the heating and cooling system so as to be entirely heated or cooled thereby; and wherein said first plate includes a surface that is adapted to be brought into abutting contact with a person's skin to impart heating or cooling to the same.

24. The device as defined in claim 23, wherein the heating and cooling system includes a Peltier effect device that is thermally connected to the first plate and a heat sink that is thermally connected to the Peltier effect device.
